(19) United States
(12) Patent Application Publication
(10) Pub. No.: US 2007/0161796 A1
(43) Pub. Date: Jul. 12, 2007

(54) Drying methods of montelukast sodium by azeotropic removal of the solvent

(76) Inventors: Ilan Kor-Sade, Shoham (IL); Amir Gold, Herzelia (IL); Claude Singer, Kfar Saba (IL); Ziv Dee-Noor, Haifa (IL); Omer Malachi, Rehovot (IL)

Correspondence Address: KENYON & KENYON LLP, ONE BROADWAY, NEW YORK, NY, 10004, US

(21) Appl. No.: 11/601,113

(22) Filed: Nov. 16, 2006

Related U.S. Application Data
(60) Provisional application No. 60/737,730, filed on Nov. 16, 2005.
(60) Provisional application No. 60/753,126, filed on Dec. 21, 2005.

(51) Int. Cl. C07D 215/18

(52) U.S. Cl. 546/180

(57) ABSTRACT

The invention encompasses amorphous montelukast sodium having less than about 50 ppm heptane or less than about 100 ppm hexane, less than about 150 ppm toluene, and less than about 2500 ppm C₁-C₅ alcohols, as well as processes for its preparation.

### CROSS-REFERENCE TO RELATED APPLICATIONS

This application claims the benefit of priority to U.S. provisional application Ser. Nos. 60/737,730, filed Nov. 16, 2005, and 60/753,126, filed Dec. 21, 2005, hereby incorporated by reference.

### FIELD OF THE INVENTION

The invention encompasses amorphous montelukast sodium having less than about 50 ppm heptane or less than about 100 ppm hexane, less than about 150 ppm toluene, and less than about 2500 ppm C₁-C₅ alcohols, as well as processes for its preparation.

### BACKGROUND OF THE INVENTION

Montelukast is a selective, orally active leukotriene receptor antagonist that inhibits the cysteinyl leukotriene CysLT₁ receptor. Leukotrienes are associated with the inflammation and constriction of airway muscles and the accumulation of fluid in the lungs. Montelukast sodium is a useful therapeutic agent for treating respiratory diseases such as asthma and allergic rhinitis.

The chemical name for montelukast sodium is [R-(E)]-1-[[[1-[3-[2-(7-chloro-2-quinolinyl)ethenyl]phenyl]-3-[2-(1-hydroxy-1-methylethyl)phenyl]propyl]thio]methyl]cyclopropaneacetic acid, monosodium salt. Montelukast sodium salt is represented by the following chemical structure:

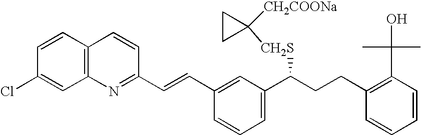

Montelukast sodium as marketed is a hygroscopic, optically active, white to off-white powder. Analysis of montelukast SINGULAIR® tablets (10 mg) demonstrated that the tablets contain toluene and DMF as residual solvents. See infra Example 5. Montelukast sodium is freely soluble in methanol, ethanol, and water and practically insoluble in acetonitrile. See Pharmacopeial Forum, 24(6) (1998), p. 7161; Summary Basis of Approval NDA 20-829, which is available at http://www.fda.gov/cder/foi/nda/98/020829s000_SingulairTOC.htm (Nov. 16, 2006).

In Example 161, Step 6, U.S. Pat. No. 5,565,473 (“'473 patent”) discloses a process for preparing montelukast sodium, wherein the montelukast sodium is obtained as oil that is then dissolved in water and freeze-dried. '473 patent, col. 81, ll. 34-36; col. 79, ll. 14-16.

The amorphous form of montelukast sodium is disclosed in U.S. Pat. No. 6,320,052 (“'052 patent”). The '052 patent cites EP 480,717, U.S. Pat. No. 5,270,324 and EP 604,114 as disclosing processes of synthesizing leukotriene antagonists. '052 patent, col. 1, ll. 44-53. The '052 patent characterizes these references as preparing amorphous forms of leukotriene antagonists and asserts that such amorphous forms are “not ideal for pharmaceutical formulation.” Id. at col. 1, ll. 54-67. The '052 patent also characterizes the processes of synthesizing leukotriene antagonists disclosed in the references as “not particularly suitable for large-scale production” because of the “tedious chromatographic purification” technique required and because the “product yields are low.” Id. In Example 8 of the '052 patent, montelukast sodium salt is crystallized from a solution of montelukast sodium in toluene and water, by the addition of acetonitrile (“ACN”) with seeding. '052 patent, col. 16, l. 62 to col. 17, l. 21.

PCT publication No. WO 03/066598 (“WO '598”) discloses a method for preparing anhydrous amorphous montelukast sodium by dissolving montelukast free acid in toluene; converting the montelukast free acid to montelukast sodium in the presence of methanol; concentrating the reaction mixture and adding hexane, n-heptane, or cyclohexane; isolating the montelukast sodium; and drying the montelukast sodium.

PCT publication No. WO 2005/074893 (“WO '893”) discloses the preparation of amorphous montelukast sodium by spray-drying a solution of montelukast sodium in solvents including acetone, C₁-C₃ alcohols, such as ethanol, water, and mixtures thereof.

None of the above-described methods for preparing amorphous montelukast sodium can provide the product with residual solvent levels low enough to comply with the International Conference on Harmonisation of Technical Requirements for Registration of Pharmaceuticals for Human Use (“ICH”) guidelines. For example, according to the ICH Q3C(R3) guidelines (November 2005), drug products should contain less than about 290 ppm hexane, less than about 890 ppm toluene, less than about 3000 ppm methanol, and less than about 5000 ppm ethanol. As discussed above, amorphous montelukast sodium has been prepared by processes involving solvents such as n-heptane or hexane, toluene, and ethanol. Removal of these solvents may require drying of the montelukast sodium at high temperature (e.g., 85° C. and above).

There is a need in the art for a new method of preparing amorphous montelukast sodium with a residual solvent content that will comply with the ICH guidelines.

### SUMMARY OF THE INVENTION

The invention encompasses a process for preparing amorphous montelukast sodium comprising: combining wet montelukast sodium having at least one residual solvent selected from the group consisting of heptane, hexane, toluene, methanol, and C₂-C₅ alcohols, and a solvent to form a reaction mixture, wherein the heptane is present in an amount of more than about 5000 ppm, the hexane is present in an amount of more than about 290 ppm, the toluene is present in an amount of more than about 890 ppm, the methanol is present in an amount of more than about 3000 ppm and the C₂-C₅ alcohol is present in an amount of more than about 5000 ppm, and the solvent forms an azeotrope with at least one of the residual solvents; and removing the azeotrope from the reaction mixture to obtain a precipitate of amorphous montelukast sodium, wherein the amorphous montelukast sodium has less than about 5000 ppm heptane or less than about 299 ppm hexane, less than about 890 ppm toluene, less than about 3000 ppm methanol, and less than about 5000 ppm C₂-C₅ alcohols.

The invention also encompasses amorphous montelukast sodium having less than about 50 ppm heptane or less than about 100 ppm hexane, less than about 150 ppm toluene, and less than about 2500 ppm C₁-C₅ alcohols.

The invention also encompasses a pharmaceutical composition comprising amorphous montelukast sodium having less than about 50 ppm heptane or less than about 100 ppm hexane, less than about 150 ppm toluene, and less than about 2500 ppm C₁-C₅ alcohols and at least one pharmaceutically acceptable excipient.

The invention also encompasses a process for preparing the pharmaceutical composition of claim 23 comprising combining the amorphous montelukast sodium and the pharmaceutically acceptable excipient.

The invention also encompasses a method of treating respiratory diseases comprising administering a therapeutically effective amount of the pharmaceutical composition of claim 23 to a patient in need of treatment thereof.

The invention also encompasses use of amorphous montelukast sodium having less than about 50 ppm heptane or less than about 100 ppm hexane, less than about 150 ppm toluene, and less than about 2500 ppm C₁-C₅ alcohols in the manufacture of a medicament for the treatment of respiratory diseases.

### BRIEF DESCRIPTION OF THE FIGURES

FIG. 1. Mass spectrum of SINGULAIR® tablet

### DETAILED DESCRIPTION OF THE INVENTION

Exposure of montelukast sodium to high temperatures, as described in the processes of the prior art, may induce degradation, such as by oxidation, which also may cause a color change. Further, the use of high temperature is undesirable for use on an industrial scale due to the increased cost involved.

The invention encompasses a process for preparing amorphous montelukast sodium by azeotropic evaporation, which produces amorphous montelukast sodium with residual solvent levels low enough to comply with ICH guidelines, without the need for drying the product at high temperature.

As used herein, unless otherwise defined, the term “azeotrope” refers to a liquid mixture of at least two components that boils at constant temperature without change in composition. The temperature at which the azeotrope boils differs from the boiling points of its individual components.

As used herein, unless otherwise defined, the term “dry,” when referring to montelukast sodium, means montelukast sodium having less than about 5000 ppm heptane or less than about 299 ppm hexane, less than about 890 ppm toluene, less than about 3000 ppm methanol, and less than about 5000 ppm C₂-C₅ alcohols.

As used herein, unless otherwise defined, the term “wet,” when referring to montelukast sodium, means montelukast sodium having at least one residual solvent selected from the group consisting of heptane, hexane, toluene, methanol, and C₂-C₅ alcohols, wherein the heptane is present in an amount of more than about 5000 ppm, the hexane is present in an amount of more than about 299 ppm, the toluene is present in an amount of more than about 890 ppm, the methanol is present in an amount of more than about 3000 ppm, and the C₂-C₅ alcohol is present in an amount of more than about 5000 ppm. “Wet” montelukast sodium may be in the form of a precipitate or a slurry.

As used herein, unless otherwise defined, the term “residual solvents” refers to a solvent selected from the group consisting of heptane, hexane, toluene, C₁-C₅ alcohols, and mixtures thereof.

As used herein, unless otherwise defined, the term “vacuum” refers to a pressure of less than about 760 mm Hg, preferably less than about 300 mm Hg, and more preferably less than about 50 mm Hg.

The invention encompasses amorphous montelukast sodium having less than about 50 ppm heptane or less than about 100 ppm hexane, less than about 150 ppm toluene, and less than about 2500 ppm C₁-C₅ alcohols.

The invention also encompasses processes for preparing amorphous montelukast sodium by azeotropic removal of residual solvent to obtain a dry powder of amorphous montelukast sodium. The azeotropic removal is used to remove residual solvents from wet montelukast sodium. The process comprises combining wet montelukast sodium and a solvent to form a reaction mixture, wherein the solvent forms an azeotrope with at least one of the residual solvents, and removing the azeotrope from the reaction mixture to obtain a precipitate of dry amorphous montelukast sodium.

The starting wet montelukast sodium may be prepared according to processes known to those of skill in the art. One such process is disclosed in U.S. application Ser. No. 11/481,877, filed Jul. 5, 2006 (“'877 application”). See infra Example 4. The '877 application discloses a process for preparing wet montelukast sodium by dissolving montelukast di-N-propyl amine salt in toluene, adding sodium tert-butoxide, adding heptane to obtain a precipitate of montelukast sodium, filtering and drying the precipitate to obtain wet montelukast sodium. Typically, the wet montelukast sodium obtained after filtration contains about 20-50% by weight of residual solvents. Typically, the wet montelukast sodium obtained after drying contains about 1200-5000 ppm toluene.

Preferably, the starting montelukast sodium is wet. More preferably, the montelukast sodium contains no more than about 50% by weight of residual solvents.

Preferably, the solvent is a C₁-C₅ alcohol, ketone, dichloromethane (“DCM”), or water. Preferably, the alcohol is methanol, ethanol, propanol, isopropanol, butanol, t-butanol, or pentanol. Preferably, the ketone is acetone, methyl isobutyl ketone (“MIBK”), or methylethyl ketone (“MEK”).

Preferably, the solvent is present in an amount of at least about 1.5 milliliters per gram of wet montelukast sodium and more preferably at least about 3 milliliters per gram of wet montelukast sodium. Even more preferably, the solvent is present in an amount of more than 2 volumes per gram of wet montelukast sodium.

Preferably, the wet montelukast sodium and the solvent are heated to form the reaction mixture. Preferably, the wet montelukast sodium and solvent are heated at a temperature of less than about 70° C., more preferably at a temperature of less than about 60° C., even more preferably at a temperature of about 35° C. to about 50° C., and most preferably at a temperature of about 40° C. to form the mixture. Preferably, the mixture is a solution or a suspension.

Optionally, the wet montelukast sodium may be further purified by any method known to one of skill in the art. Such methods include, but are not limited to, treating the reaction mixture of wet montelukast sodium and solvent with active carbon.

Preferably, the solvent removal is by evaporation. Preferably, the evaporation is performed under vacuum at a temperature sufficient to evaporate the azeotrope. The evaporation pressure can be determined by one of skill in the art based upon the composition of the azeotrope to be evaporated with little or no experimentation. Preferably, the temperature is less than about 85° C., more preferably less than about 70° C., and most preferably less than about 60° C. Preferably, the evaporation is performed in a dryer. The dryer may be an agitated vacuum dryer, tumbling vacuum dryer, or static vacuum dryer. Preferably, the agitated vacuum dryer is a pan dryer, pedal dryer, single or twin shaft high viscosity processors (such as Discotherm B produced by List A/G), thin-film evaporator, or conic dryer (such as Ekato VPT-3 conical agitated vacuum dryer). Typically, the tumbling vacuum dryer is a double cone dryer or rotary evaporator. Typically, the static vacuum dryer is a tray dryer. See A. S. Mujumdar, Handbook of Industrial Drying, 2d ed. (Marcel Dekker, Inc. 1995). Preferably, the evaporation is performed in a tray dryer or a conic dryer.

Preferably, the obtained amorphous montelukast sodium is dry. More preferably, the montelukast sodium contains less than about 50 ppm heptane or less than about 100 ppm hexane, less than about 150 ppm toluene, and less than about 2500 ppm C₁-C₅ alcohols. The amorphous montelukast sodium obtained may be analyzed to determine the amounts of solvents present using any method known to one of skill in the art. Suitable methods include, but are not limited to, headspace gas chromatography.

Preferably, the process may further comprise breaking the dry precipitate of amorphous montelukast sodium to obtain a powder of dry amorphous montelukast sodium. The dry precipitate may be broken down manually or by agitating until a powder of amorphous montelukast sodium is obtained.

TABLE 1
The amount of solvents present in amorphous montelukast
sodium after drying in a tray dryer.

| Drying | | | | | |
|---|---|---|---|---|---|
| Time | Temperature | n-Heptane | t-Butanol | Toluene | Methanol |
| (hours) | (° C.) | (ppm) | (ppm) | (ppm) | (ppm) |
| 0 | | Not relevant | | | |
| 6 | 50° C. | Not | 400 | Not | 2000 |
| | | detected | | detected | |
| 22 | 50° C. | Not | 400 | Not | 900 |
| | | detected | | detected | |
| 27 | 50° C. | Not | 400 | Not | 500 |
| | | detected | | detected | |

The invention further provides a process for preparing amorphous montelukast sodium by azeotropic removal of residual solvent in a conic dryer to obtain a powder of amorphous montelukast sodium. The process comprises: putting wet montelukast sodium in a conic dryer, adding a solvent that forms an azeotrope with at least one of the residual solvents to form a reaction mixture, and removing the azeotrope from the reaction mixture to obtain a powder of amorphous montelukast sodium.

Preferably, the montelukast sodium is wet. More preferably, the montelukast sodium contains no more than about 50% by weight of residual solvents.

Preferably, the removal is by evaporation.

The wet montelukast sodium and the solvents used in this process are as described above. The temperatures and the pressure in the conic dryer are also as described above. Optionally, the reaction mixture may be treated with active carbon. The process continues until consuming the reaction mixture.

Preferably, the conic dryer is a conical agitated vacuum dryer such as Ekato VPT-3.

Preferably, the obtained amorphous montelukast sodium is dry. More preferably, the montelukast sodium contains less than about 50 ppm heptane or less than about 100 ppm hexane, less than about 150 ppm toluene, and less than about 2500 ppm C₁-C₅ alcohols.

TABLE 2
The amount of solvents present in amorphous montelukast
sodium after drying in a conic dryer.

| Drying | | | | | |
|---|---|---|---|---|---|
| Time | Temperature | n-Heptane | t-Butanol | Toluene | Methanol |
| (hours) | (° C.) | (ppm) | (ppm) | (ppm) | (ppm) |
| 12 | 55° C. | 0.2 | 30 | 70 | 600 |

The amorphous montelukast sodium of the invention may be formulated into pharmaceutical compositions and dosage forms to be used in treating respiratory diseases.

The pharmaceutical compositions comprise amorphous montelukast sodium having less than about 50 ppm heptane or less than about 100 ppm hexane, less than about 150 ppm toluene, and less than about 2500 ppm C₁-C₅ alcohols and at least one pharmaceutically acceptable excipient.

The pharmaceutical compositions may be prepared by a process comprising combining the amorphous montelukast sodium and the pharmaceutically acceptable excipient. The amorphous montelukast sodium may be prepared by the above-described process.

The invention also encompasses pharmaceutical formulations comprising the amorphous montelukast sodium of the present invention, and pharmaceutically acceptable excipient.

The invention further encompasses a process for preparing a pharmaceutical formulation comprising combining amorphous montelukast sodium of the present invention with at least one pharmaceutically acceptable excipient.

The invention further encompasses the use of amorphous montelukast sodium of the present invention for the manufacture of a pharmaceutical composition.

Pharmaceutical compositions containing amorphous montelukast sodium can optionally contain a mixture of other form(s) of montelukast. In addition to the active ingredient(s), the pharmaceutical formulations can contain one or more excipients. The active ingredient and excipient(s) can be formulated into compositions and dosage forms according to methods known in the art. Excipients are added to the formulation for a variety of purposes.

Pharmaceutical compositions can be prepared as medicaments to be administered orally, parenterally, rectally, transdermally, bucally, or nasally. Suitable forms for oral administration include solid dosage forms like tablets, powders, capsules, suppositories, sachets, troches and lozenges, as well as liquid syrups, suspensions, and elixirs. Suitable forms of parenteral administration include an aqueous or non-aqueous solution or emulsion, while for rectal administration suitable forms for administration include suppositories with hydrophilic or hydrophobic vehicle. For topical administration, the invention provides suitable transdermal delivery systems known in the art, and for nasal delivery, there are provided suitable aerosol delivery systems known in the art.

Selection of excipients and the amounts to use can be readily determined by the formulation scientist based upon experience and consideration of standard procedures and reference works in the field. For example, diluents increase the bulk of a solid pharmaceutical composition, and can make a pharmaceutical dosage form containing the composition easier for the patient and care giver to handle. Diluents for solid compositions include, for example, microcrystalline cellulose (e.g., AVICEL®), microfine cellulose, lactose, starch, pregelitinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., EUDRAGIT®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include acacia, alginic acid, carbomer (e.g., CARBOPOL®), carboxymethylcellulose sodium, dextrin, ethyl cellulose, gelatin, guar gum, hydrogenated vegetable oil, hydroxyethyl cellulose, hydroxypropyl cellulose (e.g., KLUCEL®), hydroxypropyl methyl cellulose (e.g., METHOCEL®), liquid glucose, magnesium aluminum silicate, maltodextrin, methylcellulose, polymethacrylates, povidone (e.g., KOLLIDON®, PLASDONE®), pregelatinized starch, sodium alginate, and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g., AC-DI-SOL®, PRIMELLOSE®), colloidal silicon dioxide, croscarmellose sodium, crospovidone (e.g., KOLLIDON®, POLYPLASDONE®), guar gum, magnesium aluminum silicate, methyl cellulose, microcrystalline cellulose, polacrilin potassium, powdered cellulose, pregelatinized starch, sodium alginate, sodium starch glycolate (e.g., EXPLOTAB®), and starch.

Glidants can be added to improve the flowability of a non-compacted solid composition and to improve the accuracy of dosing. Excipients that can function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that can be included in the composition of the invention include maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

In liquid pharmaceutical compositions, the active ingredient and any other solid excipients are dissolved or suspended in a liquid carrier such as water, vegetable oil, alcohol, polyethylene glycol, propylene glycol, or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the invention can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, and xanthan gum.

Sweetening agents such as sorbitol, saccharin, sodium saccharin, sucrose, aspartame, fructose, mannitol, and invert sugar can be added to improve the taste.

Preservatives and chelating agents such as alcohols, sodium benzoate, butylated hydroxy toluene, butylated hydroxyanisole, and ethylenediamine tetraacetic acid can be added at levels safe for ingestion to improve storage stability.

According to the invention, a liquid composition can also contain a buffer such as gluconic acid, lactic acid, citric acid or acetic acid, sodium gluconate, sodium lactate, sodium citrate or sodium acetate.

The solid compositions of the invention include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, the most preferred route of the invention is oral. The dosages can be conveniently presented in unit dosage form and prepared by any of the methods well-known in the pharmaceutical arts.

A composition for tableting or capsule filling can be prepared by wet granulation. In wet granulation, some or all of the active ingredients and excipients in powder form are blended and then further mixed in the presence of a liquid, typically water, that causes the powders to clump into granules. The granulate is screened and/or milled, dried and then screened and/or milled to the desired particle size. The granulate can then be tabletted, or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the invention can comprise any of the aforementioned blends and granulates that were described with reference to tableting, however, they are not subjected to a final tableting step. A capsule dosage form contains the composition, preferably a powdered or granulated solid composition of the invention, within either a hard or soft shell. The shell can be made from gelatin and optionally contain a plasticizer such as glycerin and sorbitol, and an opacifying agent or colorant.

The pharmaceutical compositions of the invention can be used to treat respiratory diseases such as asthma and allergic rhinitis in a mammal, preferably a human, by administering a therapeutically effective amount of the pharmaceutical composition to a mammal in need thereof.

Having thus described the invention with reference to particular preferred embodiments and illustrative examples, those in the art can appreciate modifications to the invention as described and illustrated that do not depart from the spirit and scope of the invention as disclosed in the specification. The following examples are set forth to aid in understanding the invention but are not intended to, and should not be construed to, limit its scope in any way. The examples do not include detailed descriptions of conventional methods. Such methods are well known to those of ordinary skill in the art and are described in numerous publications.

### EXAMPLES

Methods

Headspace Gas Chromatoraphic (GC-HS) Method

An automatic headspace gas-chromatographic system was adopted for determination of the residual solvents in montelukast sodium.

The sample is dissolved in dimethyl sulfoxide (about 100 mg in 1 mL). The dissolved residual solvents should be determined by static headspace gas chromatographic, external standard method.

Equipment:

Gas chromatograph: Model HP-6890

Headspace sampler: Combi Pal (CTC Analytics)

Analytical balance: ±0.01 mg Model AT-201 Mettler

Micro syringe: 250 μL

Column: HP-Fast Residual Solvent Column, 30m×0.53 mm×1 μm (Cat. No. 19095V-420-Agilent) or equivalent.

Carrier gas: Helium about 3.5 psi, constant pressure (about 5 mL/min. at 40° C.).
Injection mode: Headspace split mode.
Split Ratio: 1:5 by using COMBI PAL (CTC Analytics) headspace sampler (gas-syringe technique)
Detector: Flame Ionization Detector.
Make up gas: Helium, about 25 mL/min.
Temperature: Injector: 180° C.
   Detector: 260° C.
Make up gas: Helium, about 25 mL/min.
Temperature: Injector: 180° C.
   Detector: 260° C.
   Oven program: Initial temperature: 40° C.
      Initial time: 3.0 min.
   Rate Final temp. Final time
   15° C./min. 140° C. 2.0 min
      CombiPAL (CTC Analytics) Headspace sampler (Gas-syringe technique)

Syringe: 2.5 mL

Sample volume: 1 mL

Incub. Temperature: 80° C.

Incub. Time: 35 min.

Agi Speed: 500 rpm

Agi on time: 5s

Agi off time: 5s

Syringe temp.: 100° C.

Fill speed: 300 μL/s

Pull up del.: 1 s

Inject speed: 800 μL/s

Pre Inj. del.: 0 s

Post inj. del.: 1.5s

Syr. Flushing: 2.5 min.

G.C. run time: 22 min.

Standard Solution Preparation

Weigh accurately analytical standards into a volumetric flask containing dimethylsulfoxide (“DMSO”). Fill to volume with DMSO and mix.

Analysis of Standard Solutions of Solvent

Using a ±0.01 mg Model AT-201 Mettler analytical balance, weigh accurately analytical standard of solvent into a volumetric flask containing dimethylsulfoxide (“DMSO”). Fill to volume with DMSO and mix to form standard solution of the solvent. Transfer the standard solution into a 20 ml headspace vial. Seal the vial with a septum crimp cap and test according to the GC-HS conditions described above.

Analysis of Montelukast Sodium Samples

Using a ±0.01 mg Model AT-201 Mettler analytical balance, accurately weigh 100 mg of montelukast sodium sample and transfer the sample into a 20 ml headspace vial. Add 1 ml dimethylsulfoxide to the vial, immediately seal with a septum crimp cap and mix gently. Analyze the sample according to the GC-HS conditions described above.

Calculations

Calculate the concentration in parts per million (“ppm”) of solvents present in a montelukast sodium sample using the following formula:

$$\begin{matrix}ppm solvent = \frac{{r}_{Sp 1} \times {C}_{Std} \times 1}{{r}_{Std} \times {W}_{Sp 1}} = \frac{{r}_{Sp 1} \times 1}{\overline{{RF}_{Std}} \times {W}_{Sp 1}} \\ {r}_{Sp 1} and {r}_{Std} = solvent peak area in sample solution chromatogram \\ ( {r}_{Sp 1} ) and in standard solution chromatogram ( {r}_{Std} ) \\ respectively . \\ {C}_{Std} = solvent concentration in injected standard \\ solutions in µg \text{/} mL \\ {W}_{Sp 1} = weight of sample in g . \\ \overline{R . F} {.}_{Std} = \frac{{r}_{Std}}{{C}_{Std}} = Average standard response factor .\end{matrix}$$

X-Ray Diffraction

X-ray powder diffraction data were obtained by methods known in the art using a SCINTAG powder x-ray diffractometer model X'TRA equipped with a solid state detector. Copper radiation of 1.5418Å was used. A round aluminum sample holder with round zero background quartz plate, with cavity of 25 (diameter)*0.5 (depth) mm.

Scanning parameters:

Range: 2-40 degrees two-theta (±0.2 degrees two-theta)

### Example 1

### Conic Vacuum Dryer

Wet cake (from toluene, heptane, t-butanol and THF) of montelukast sodium (250 g) was dissolved in methanol (750 ml) at 45° C. The solution was treated with active carbon (12.5 g) and then heated under continuous stirring in a controlled conical vacuum dryer (Ekato VPT3).

Methanol was removed by azeotropic evaporation at a jacket temperature of 50° C., a pressure of 200-250 mbar, and an agitator speed of 70 rpm until the solution became viscous and the material temperature was 44° C. Then the pressure was maintained at 250-230 mbar and the agitator speed was set to 50 rpm until the viscous material dried to powder. As the viscous material was drying, the amorphous montelukast sodium became swollen, forming the solid powder. The sample was analyzed by x-ray diffraction (“XRD”). Purely amorphous form of montelukast sodium was obtained. The residual solvent content of the obtained product was: n-Heptane: Not detected, Toluene=Not detected, t-BuOH=400 ppm, Methanol=900 ppm—after 22 hrs of drying at 50 degrees Celsius.

### Example 2

### Tray Drying

Wet cake (from toluene, heptane, t-butanol and THF) of montelukast sodium (250 g) was dissolved in methanol (750 ml) at 45° C. The solution was treated with active carbon (12.5 g) and then evaporated under vacuum (pressure≦300 mbar) in a tray dryer. The material was dried and the solvents were removed by azeotropic evaporation at 45° C. The sample was analyzed by XRD. Purely amorphous form of montelukast sodium was obtained.

### Example 3

### Drying by Rotary Evaporator

Wet cake (from toluene, heptane, t-butanol and THF) of montelukast sodium (250 g) was dissolved in methanol (750 ml) at 45° C. The solution was treated with active carbon (1.5 g) and then evaporated under vacuum (pressure≦300 mbar) in a rotary evaporator. The material was dried and the solvents were removed by azeotropic evaporation at 45° C. The sample was analyzed by XRD. Purely amorphous form of montelukast sodium was obtained.

### Example 4

### The preparation of [R-(E)]-1-[[[3-[2-(7-chloro-2-quinolinyl)ethenyl]phenyl]-3-[2-(1-hydroxy-1-methylethyl)phenyl]propyl]thio]methyl]cyclopropaneacetic sodium salt According to U.S. application Ser. No. 11/481,877

A 500 ml flask equipped with mechanic stirrer was charged with toluene (225 ml) and montelukast di-n-propylamine salt (45 g). The suspension was stirred at ambient temperature for 30 minutes. Sodium tert-butoxide (6.5 g) was added to the suspension, and the reaction mixture was stirred at 30-40° C. for 30 minutes. Active carbon (2 g) was added, and the solution was filtered over active carbon.

The mixture was added portionwise to a flask containing heptane (630 ml) to form a precipitate, and the mixture was further stirred at ambient temperature for 1 hour.

The montelukast sodium salt crystals were collected by filtration, washed with heptane, and dried at 45° C. under reduced pressure. Montelukast sodium (32 g) was obtained as an amorphous material containing greater than 1% water. The amount of [R-(E)]-1-[[[1-[3-[2-(7-chloro-2-quinolinyl)ethenyl]phenyl]-3-[2-(1-propenyl)phenyl]propyl]thio]methyl]cyclopropaneacetic acid (“MLK-D”) was reduced to an undetectable level.

### Example 5

### Experimental Conditions and Results of an Analysis of the SINGULAIR® Tablets

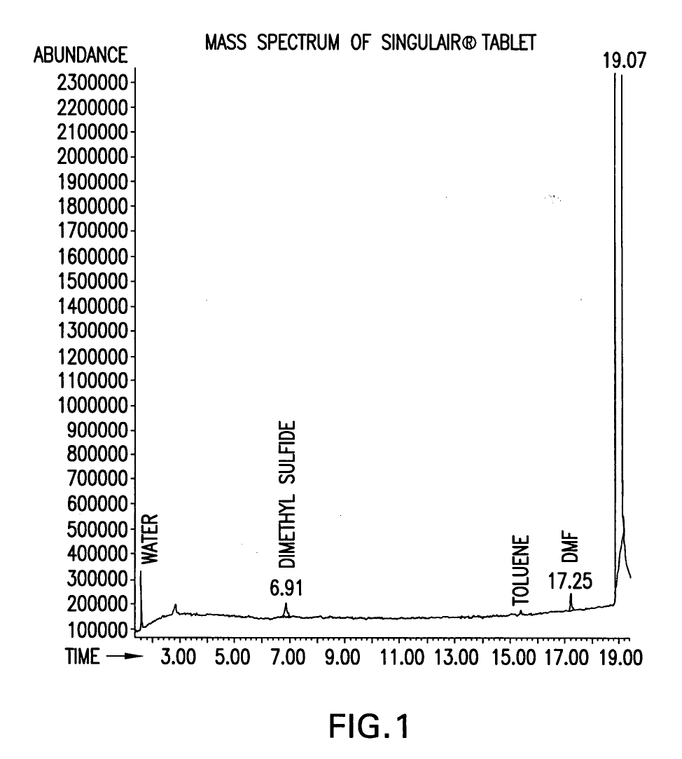

A sample of SINGULAIR® was tested by GC-MS in order to identify residual solvents. Rtx-1301 60m×0.32 mm×1.5μ column was used. To enhance sensitivity, three SINGULAIR® tablets were ground, and analyzed both as a dry sample and in solution in 1 mL dimethylsulfoxide. Both preparations were equilibrated at 80° C. for an hour, and 1 mL headspace injected manually in Scan mode (m/z=19-200). The mass spectrum is illustrated in FIG. 1.

We claim:

1. A process for preparing amorphous montelukast sodium comprising:
a) combining wet montelukast sodium having at least one residual solvent selected from the group consisting of heptane, hexane, toluene, methanol, and C₂-C₅ alcohols, and a solvent to form a reaction mixture,
wherein the heptane is present in an amount of more than about 5000 ppm, the hexane is present in an amount of more than about 290 ppm, the toluene is present in an amount of more than about 890 ppm, the methanol is present in an amount of more than about 3000 ppm and the C₂-C₅ alcohol is present in an amount of more than about 5000 ppm, and the solvent forms an azeotrope with at least one of the residual solvents; and
b) removing the azeotrope from the reaction mixture to obtain a precipitate of amorphous montelukast sodium, wherein the amorphous montelukast sodium has less than about 5000 ppm heptane or less than about 299 ppm hexane, less than about 890 ppm toluene, less than about 3000 ppm methanol, and less than about 5000 ppm C₂-C₅ alcohols.

2. The process of claim 1, wherein the amorphous montelukast sodium has less than about 50 ppm heptane or less than about 100 ppm hexane, less than about 150 ppm toluene, and less than about 2500 ppm C₁-C₅ alcohols.

3. The process of claim 1, wherein the solvent is a C₁-C₅ alcohol, ketone, dichloromethane, or water.

4. The process of claim 1, wherein the solvent is acetone, methyl isobutyl ketone, or methylethyl ketone.

5. The process of claim 1, wherein the solvent is present in an amount of at least about 1.5 milliliters per gram of the wet montelukast sodium.

6. The process of claim 1, wherein the solvent is present in an amount of at least about 3 milliliters per gram of the wet montelukast sodium.

7. The process of claim 1, wherein the solvent is present in an amount of more than 2 volumes per gram of the wet montelukast sodium.

8. The process of claim 1, wherein the reaction mixture is heated at a temperature of less than about 70° C. prior to removing the azeotrope.

9. The process of claim 1, wherein the reaction mixture is heated at a temperature of less than about 60° C. prior to removing the azeotrope.

10. The process of claim 1, wherein the reaction mixture is heated at a temperature of about 35° C. to about 50° C. prior to removing the azeotrope.

11. The process of claim 1, wherein the reaction mixture is heated at a temperature of about 40° C. prior to removing the azeotrope.

12. The process of claim 1, wherein the reaction mixture is treated with active carbon prior to removing the azeotrope.

13. The process of claim 1, azeotrope is removed by evaporation.

14. The process of claim 13, wherein the evaporation is performed under vacuum at a temperature below about 85° C.

15. The process of claim 13, wherein the evaporation is performed under vacuum at a temperature below about 70° C.

16. The process of claim 13, wherein the evaporation is performed under vacuum at a temperature below about 60° C.

17. The process of claim 13, wherein the evaporation is performed in a dryer.

18. The process of claim 17, wherein the dryer is an agitated vacuum dryer, a tumbling vacuum dryer, or a static vacuum dryer.

19. The process of claim 17, wherein the dryer is a tray dryer or a conic dryer.

20. Amorphous montelukast sodium having less than about 50 ppm heptane or less than about 100 ppm hexane, less than about 150 ppm toluene, and less than about 2500 ppm C₁-C₅ alcohols.

21. A pharmaceutical composition comprising amorphous montelukast sodium and at least one pharmaceutically acceptable excipient, wherein the amorphous montelukast sodium has less than about 50 ppm heptane or less than about 100 ppm hexane, less than about 150 ppm toluene, and less than about 2500 ppm C₁-C₅ alcohols

22. A process for preparing the pharmaceutical composition of claim 21 comprising combining the amorphous montelukast sodium and the pharmaceutically acceptable excipient.

23. A method of treating respiratory diseases comprising administering a therapeutically effective amount of the pharmaceutical composition of claim 21 to a patient in need of treatment thereof.
